(19) United States
(12) Patent Application Publication
(10) Pub. No.: US 2007/0027457 A1
(43) Pub. Date: Feb. 1, 2007

(54) Band blade suture remover apparatus and method

(76) Inventor: Carol Margaret Walborn, Chapel Hill, NC (US)

Correspondence Address: Carol Walborn, 107 Saratoga Trail, Chapel Hill, NC, 27516, US

(21) Appl. No.: 11/488,358

(22) Filed: Jul. 18, 2006

Related U.S. Application Data
(60) Provisional application No. 60/699,643, filed on Jul. 18, 2005.

(51) Int. Cl. A61B 17/10

(52) U.S. Cl. 606/138

(57) ABSTRACT

Apparatuses and methods for grasping, holding, cutting, and removing knotted sutures from the surface of the skin of humans and animals are disclosed, wherein the band blade suture remover apparatus performs suture removal in a one-handed, single motion to minimize pain to the patient, eliminate fearful-appearing surgical apparatus for children, and reduce time involved for suture removal procedures. The band blade suture remover apparatus for the cutting and removal of sutures is provided comprising of a circular housing with trigger and hand grip that encases an elongate member of thin steel with an angled cutting blade at one end for cutting the suture and a v-shaped slot at the opposite end of the elongate member for grasping and holding sutures during the cutting and removal procedure. The elongate member is bent along its longitude in a hoop-like fashion to form a circular band blade.

### RELATED APPLCATIONS

This application claims the benefit of U.S. Provisional Patent Application Ser. No. 60/699,643, filed Jul. 18, 2005; the disclosure of which is incorporated herein by reference in its entirety.

### TECHNICAL FIELD

The subject matter herein relates generally to medical devices. More particularly, the present subject matter relates to apparatuses and methods for holding, cutting, and removing knotted sutures wherein a steel, circular band with a cutting edge (blade) on one end and a slot for grasping and holding suture material on the opposite end, incorporated into certain embodiments, can easily and quickly remove knotted sutures from the surface of the skin of humans and animals.

### BACKGROUND ART

As a result of various accidental injuries and/or medical procedures, it is often necessary and desired to employ knotted sutures at the surface of the skin to close a wound. Upon the healing of the wound, it then becomes necessary to remove the knotted sutures. The most common method to remove knotted sutures utilizes surgical scissors and forceps or tweezers.

The doctor, nurse, veterinarian or clinician (medical staff) holds the forceps/tweezers in one hand to grasp the knot of the suture at the skin's surface, pulling slightly upward or outward to separate the knot from the newly-healed wound site. With the other hand the medical staff positions the surgical scissors just below the knot to cut the suture's loop(s) that have been knotted to maintain wound closure during the healing process. The medical staff then pulls out the remaining suture material from the patient's wound with the forceps/tweezers.

Removal of knotted sutures is often painful for human patients, particularly for children, and often causes bleeding at the suture site. The greatest pain for human patients is associated with suture removal from the face and hands which contain the greatest number of nerves with pain receptors. Children are frightened by the site of sharp-pointed surgical instruments being used on their bodies. This method also creates problems the medical staff: a significant amount of time is needed; it is difficult to do on animal patients as they pull away during the most critical moments of the procedure; and the necessity to use both hands to remove the sutures, limits the ability to restrain or support the patient during the procedure.

Efforts have been made to address some of the problems with suture removal. U.S. Pat. Nos. 4,527,331 and 4,246,698 to Lasner improve the scissor design and include a suture gripping element in the scissors. A thermal suture cutting device, U.S. Pat. No. 7,048,746 to Warden addresses some of the discomfort associated with suture removal by melting the suture with a heating element.

While these prior art suture remover-cutters have limited success in addressing the problems associated with suture removal, there remains a need for a simple, single-motion, one-handed suture remover that minimizes pain for the patient and has a benign, non-surgical appearance to ease some of the frightening aspects of suture removal with children.

### SUMMARY

According to one embodiment, a band blade suture remover apparatus for the cutting and removal of sutures is provided comprising of a circular plastic housing with skin guard and hand grip that encases an elongate member of thin steel with an angled cutting blade at one end for cutting the suture and a v-shaped slot at the opposite end of the elongate member for grasping and holding suture knots. The elongate member is bent along its longitude in a hoop-like fashion to form a circular band blade. The band blade suture remover apparatus further comprises a pivoting trigger mechanism that is attached by plastic or metal rivets to the band blade by a trigger elongate member of thin steel such that it binds the band blade at its first attach point, which is % the distance of its circumference of the band blade from the cutting edge, to the trigger elongate member, which is also attached at a second attach point to the upper portion of the trigger and the housing by way of plastic or metal rivets which allow the trigger to pivot as a lever.

The band blade suture remover apparatus pivoting trigger mechanism further comprises a finger trigger, when squeezed in a pulling motion toward the hand-grip by applying finger pressure to the trigger, to bring the opposite ends of the band blade together in a closed position such that the band blade's outer and underneath surface of the v-slot end overlaps the inner and upper surface of the cutting blade in a slidingly, slicing motion for the purpose of holding, cutting, and removing the suture.

The triggering mechanism further comprises a pressure arm attached at one end to at a point on the trigger elongate member and at the other end passing through a hole in the band blade nearest the v-slot end to attach to a plastic skin guard pressure plate located underneath the cutting edge when in the closed position. The application of pressure to the trigger when closing the band blade's v-slot end over the cutting blade, simultaneously positions the skin guard underneath the cutting blade and pushes the skin guard in a downward and outward motion to gently press the skin away from the cutting blade to protect the skin and detach the suture knot from the skin for cutting and removal.

### BRIEF DESCRIPTION OF THE DRAWINGS

FIG. 1 is a side plan view of the Band Blade Suture Remover in accordance with one embodiment of the present subject matter.

FIGS. 2A and 2B is a side plan view of the band blade mechanism in the open and closed positions of the present subject matter.

FIG. 3 is a side plan view detailing various elements of the preferred embodiment of a Band Blade Suture Remover of the present subject matter.

### DETAILED DESCRIPTION

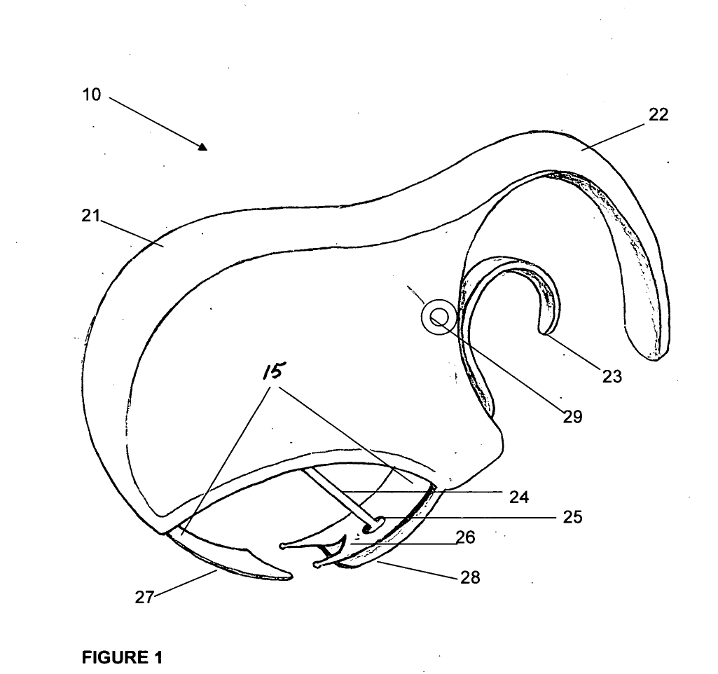

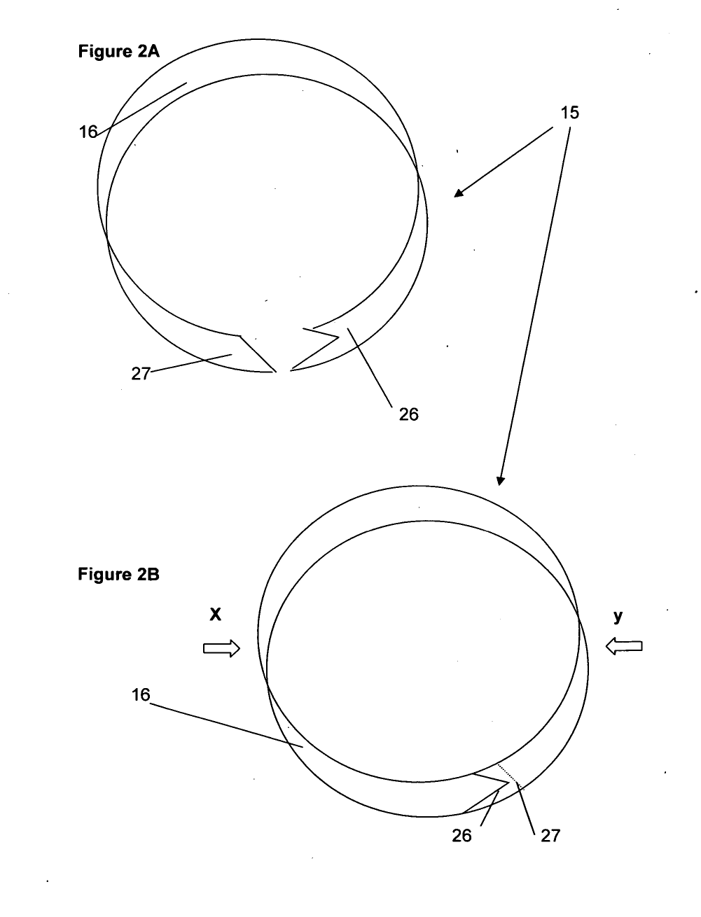

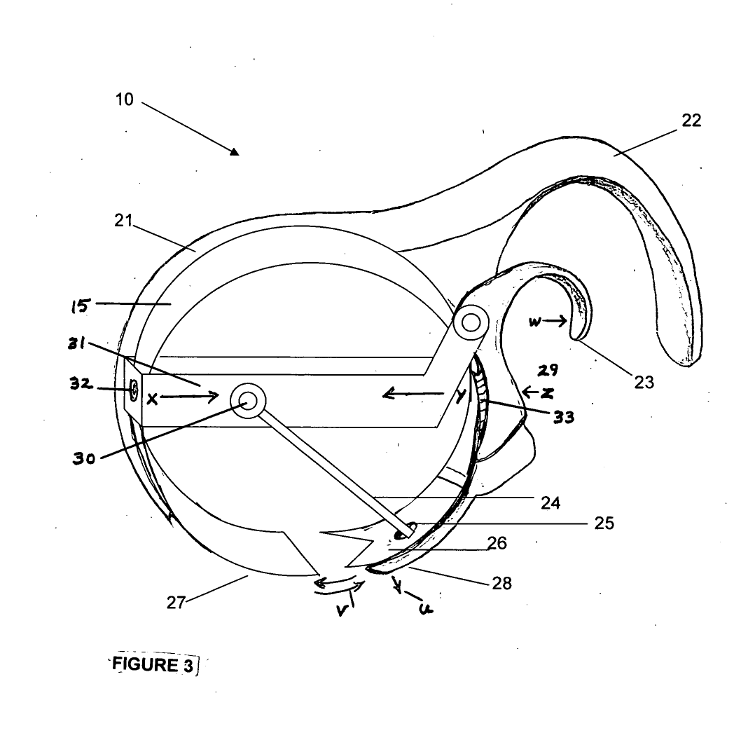

Referring now to FIG. 1, an exemplary embodiment of the band blade suture remover apparatus of the present subject matter is shown generally as 10. Band blade suture remover apparatus 10 is designed to grasp and hold a knotted suture, cut the suture, and remove the suture loop in a one-handed, single motion that minimizes pain to the patient. Band blade suture remover apparatus 10 comprises a plastic housing 21 with a skin guard 28 and hand-grip 22 that encases a circular band blade 15 (see FIGS. 2A and 3) with one end formed as an angled cutting blade 27, and the other end formed as a v-slot 26 for grasping and holding the suture. Band blade suture remover apparatus 10 further comprises a pivot trigger mechanism 23 attached to the housing 21 by way of a free-turning plastic or metal rivet 29 (see FIG. 3) so as to allow the pivot trigger 23 to be pulled by finger pressure toward the hand-grip 22 to apply pressure to the band blade in opposite directions at its mid-point along its circumference such that it closes the cutting blade of the band blade 27 slidingly between the v-slot 26 and the skin guard 28. Band blade suture remover apparatus 10 further comprises a pressure arm 24 attached at one end to the trigger mechanism elongate member 31 by way of a plastic or metal rivet 32 (see FIG. 3) and at the other end to a skin guard 28 after passing through a hole 25 in the band blade 15 nearest the v-slot 26 for the purpose of gently pressing on the patient's skin in an outward and downward motion to detach and separate the knot away from the skin prior to cutting and removal, and to protect the patient's skin.

Referring now to FIGS. 2A and 2B, the band blade 15 shown for its mechanical operation of the present subject matter and comprises an elongate member of thin steel 16 with an angled cutting blade 27 at one end for cutting the suture and a v-shaped slot 26 at the other end of the elongate member for grasping and holding suture knots. The elongate member 16 is bent along its longitude in a hoop-like fashion to form the circular band blade 15.

Referring to FIG. 2A, the band blade 15 is shown in the open or normal position with the cutting blade 27 and the v-slot apart 26 when not in use. Referring to FIG. 2B, the band blade 15 is shown in the closed position when pressure is applied in opposite directions at points x and y in the direction of the arrows, bringing the v-slot 26 slidingly over the cutting blade 27 to grasp and hold the suture knot and allow the cutting of the suture and the removal of the suture when the apparatus is lifted away from the skin.

Referring now to FIG. 3, an exemplary embodiment of the band blade suture remover apparatus of the present subject matter is shown generally as 10. Band blade suture remover apparatus 10 comprises a circular, plastic housing 21 with trigger and hand grip 22 that encases an elongate member of thin steel bent along its longitude in a hoop-like fashion to form a circular band blade 15 with an angled cutting blade at one end 27 for cutting the suture and a v-shaped slot 26 at the other end for grasping and holding suture knots. The band blade suture remover apparatus 10 further comprises a pivoting trigger mechanism 23 that is attached by plastic or metal rivets 32 to the band blade 15 by a trigger elongate member of thin steel 31 attached to the band blade 15 at its first attach point 32, ¼ the distance of the circumference of the band blade 15 from the cutting blade 27, with the trigger elongate member 31, attached at its second point to the upper portion of the trigger 23 at a pivot point in the housing 21 by way of plastic or metal rivets 29 which allow the trigger 23 to be pulled in the direction w toward the hand-grip 22, thus pulling on the attach point 32 to create pressure in one direction x and while tilting the trigger pressure plate 33 to create push Z and pressure y on the band blade 15 thus squeezing the band blade 15 into its closed position (see FIG. 2B). When the trigger 23 is operated by finger pressure, the band blade 15 moves in a closing motion v such that the outer and underneath surface of the v-slot 26 overlaps the inner and upper surface of the cutting blade 27 in a slidingly, slicing motion for the purpose of holding and cutting the suture. The triggering mechanism further comprises a pressure arm 24 attached at one end to at a point on the trigger elongate member 31 with a plastic or metal rivet 30 and at the other end passing through a hole 25 in the band blade 15 nearest the v-slot 26 to attach to a plastic skin guard pressure plate 28 located underneath the v-slot 26 and immediately underneath the cutting blade 27 when in the closed position. The application of pressure w to the trigger 23 when closing the v-slot 26 over the cutting blade 27, simultaneously positions the skin guard underneath the cutting blade 27 and pushes the skin guard 28 in a downward and outward motion u to gently press the skin away from the cutting edge 27 to protect the skin during suture removal.

What is claimed is:

1. A band blade suture remover apparatus for the cutting and removal of sutures, the band blade suture remover apparatus comprising:
a) a circular, plastic housing with trigger and hand grip that encases a hoop-like, circular band blade of thin steel with an angled cutting blade at one end for cutting the suture and a v-shaped slot at the opposite end for grasping, holding and removing sutures;
b) wherein a band blade constructed from an elongate member of thin steel with an angled cutting blade formed on one end and a v-slot formed on the other end and the elongate member is bent in an open-ended hoop-like fashion to form a circular band blade such that when pressure is applied in opposite directions to the outside circumference of the band blade at points ¼ of the circumference from the cutting blade and ¼ the circumference from the v-slot the two ends slidingly come together with the v-slot overlapping the angled blade to hold a suture knot in the slot, cut the suture loop with the angled blade, then remove the held suture by lifting the apparatus.
c) a pivoting trigger mechanism, acting as a lever, is attached at one point on the band blade by a trigger elongate member of thin steel and to another point on the upper portion of the trigger and the housing by way of plastic or metal rivets which allow the trigger to pivot in a pull-push motion such that when the trigger is squeezed by applying finger pressure in a pulling motion toward the hand-grip it brings the opposite ends of the band blade together such that the band blade's outer and underneath surface of the v-slot end overlaps the inner and upper surface of the cutting blade in slidingly, slicing motion for the purpose of holding and cutting the suture;
d) the triggering mechanism further comprises a pressure arm attached at one end to at a point on the trigger elongate member and at the other end passing through a hole in the band blade nearest the v-slot end to attach to a plastic skin guard pressure plate located underneath the cutting edge when in the closed position. The application of pressure to the trigger when closing the band blade's v-slot end over the cutting blade, simultaneously positions the skin guard underneath the cutting edge and pushes the skin guard in a downward and outward motion to gently press the skin away from the cutting edge to protect the skin and detach the suture knot from the skin for cutting.
e) a combination of metal and plastic parts to adapt the band blade suture remover apparatus as a disposable or reusable suture remover device.

2. The band blade suture remover apparatus according to claim 1 can perform suture removal in a one-handed, single motion to minimize pain to the patient, eliminate some of the frightening appearances of such surgical apparatus for children, and reduce the time involved for the suture removal procedure.

3. The band blade suture remover apparatus according to claim 1 has two sides and a perimeter to form the hollow housing, hand grip as the basic shape of the band blade suture remover apparatus encasing the pivot trigger mechanism and the band blade for holding and cutting sutures. The outer dimensions of the band blade suture remover apparatus is approximately 5 inches measured horizontally along each side from the mid-point of the circumference of the housing to the back edge of the hand grip, and measuring approximately 4 inches from the top edge of the housing along the side of the apparatus vertically to the bottom of the band blade. The housing will vary slightly in shape to accommodate the size of the trigger mechanism and the band blade. The trigger mechanism will vary slightly in shape and dimensions to optimize operation and comfort for the user. The band blade will vary slightly in size to maximize operation, comfort and safety.
